(19) United States
(12) Patent Application Publication
(10) Pub. No.: US 2009/0264864 A1
(43) Pub. Date: Oct. 22, 2009

(54) Guide wire retention and positioning apparatus

(75) Inventors: Paul S. Teirstein, La Jolla, CA (US); David Ronan, Tuam (IE); Robert F. Carmichael, JR., Needham, MA (US); Yem Chin, Burlington, MA (US)

Correspondence Address: GERALD W. SPINKS, 103 EDWARDS STREET, ABBEVILLE, LA, 70510, US

(73) Assignee: Paul S. Teirstein

(21) Appl. No.: 12/148,681

(22) Filed: Apr. 21, 2008

(51) Int. Cl. A61M 25/09; A61M 25/18

(52) U.S. Cl. 604/528; 604/533

(57) ABSTRACT

An apparatus for retaining and positioning one or more guide wires relative to an intravascular Y adaptor, having one end of a positioning arm releasably attachable to the Y adaptor and a releasable gripping mechanism at the other end of the positioning arm for gripping the guide wire. The attachable end of the positioning arm can attach to the body or the side port of the Y connector. The gripping mechanism can include a friction fitting or a clamping device. The positioning arm can be adjustable, including a telescoping section, a flexible section, or a pivotable joint.

### CROSS REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

This invention is in the field of catheters and related apparatus, used in intravascular applications.

2. Background Art

Invasive vascular procedures like balloon angioplasty and stent implantation require insertion of a catheter into the vasculature, usually in the femoral (leg) artery and directing the catheter to the vasculature in need of treatment, such as the heart. Through this catheter, a thin (for example 0.014 inch) wire called a guide wire, is introduced and threaded down the artery to be treated.

The wire is placed into the catheter through the use of a Y adaptor. The Y adaptor contains a sealable entry site so that the wire, and other treatment devices, can be introduced with a provision for sealing around the wire, so there is no bleeding back around the site of entry into the catheter. The Y adaptor also has a tubular “side port” or “side arm” that is connected to a syringe and manifold system to allow injection of radio contrast fluid down the catheter, around the guide wire, to allow x-ray visualization. This side port is typically less than one inch in length, and it terminates with a connection device to allow attachment to a tube containing the contrast fluid, and other fluids. This attachment site is typically a “Luer” connection. Luer connection systems are the standard way of attaching syringes, catheters, hubbed needles, IV tubes, and so on, to each other. They consist of round male and female interlocking tubes, slightly tapered to hold together better with just a pressure and twist fit. Luer connections can be slip fitted, called a “luer slip”, or they can have an additional outer rim of threading, called a “luer lock”, allowing them to be far more secure.

The Y adaptor, therefore serves two functions: 1) it provides an entry for guide wire and other device introduction and 2) it provides a means for contrast and other fluid injection. At times, the operator must treat or protect more than one vessel using the same guide catheter. In this circumstance, the operator passes two or more guide wires through the same Y adaptor sealable entry site. The multiple guide wires travel down the same guide catheter and then enter the vessel requiring treatment, with each wire usually entering a different vessel or branch vessel in need of treatment.

The multiple guide wires enter the guide catheter through the sealable entry site of the Y adaptor. Since the guide wires have the same point of entry at the Y adaptor, the operator must take steps to keep the wires separate. It is important to keep the wires separate for several reasons. If the wires become twisted, they will interact with one another; for instance, when the operator moves one wire, the other wire may also move. Further, different devices, such as stents, are typically passed over the guide wires; therefore, if the guide wires become twisted, advancement of the devices is hindered. Also, since different devices are passed over the different wires, the operator must take steps to identify each wire so as not to confuse which wire is going down which vessel or branch vessel. Currently, the only available method of separating guide wires is to use layers of sterile towels. However, towels are bulky and difficult to control. Towels securing guide wires also lie on the operative field and if the Y adaptor is moved, the towels stay in place, so that the guide wires may be inadvertently pulled out of the vessel.

It is an object of the present invention to provide an apparatus which will position and retain one or more guide wires, relative to an intravascular Y adaptor.

### BRIEF SUMMARY OF THE INVENTION

The invention includes a positioning arm attached to the Y adaptor, providing a retention means to secure one or more guide wires. The invention is further refined to provide a method of securing the positioning arm to any commercially available Y adaptor. The invention is further refined to provide a method of securing the positioning arm to any commercially available Y adaptor using the tubular side port as an attachment point for the invention. The invention is further refined to provide a method of securing the positioning arm to any commercially available Y adaptor using the tubular side port fitting, for example the Luer connection, as an attachment point for the invention. The invention is further refined to provide a method of securing the positioning arm to any commercially available Y adaptor using the tubular side port fitting, for example the Luer connection, as an attachment point for the invention, such that attachment of the invention does not disrupt fluid flow through the Luer connection. The invention is further refined to provide a method of securing the positioning arm to any commercially available Y adaptor using a snap-on connection to the body of the Y adaptor.

The invention is further refined by providing a retention means at the outer end of the positioning arm, to secure one or more guide wires to the positioning arm using a friction fitting material such as rubber or other material that will passively retain the guide wires. The invention is further refined by providing a retention means at the outer end of the positioning arm, to secure one or more guide wires to the positioning arm using an actively grabbing mechanism such as a spring clamp, a slidable locking device, a screw lock or another active securing device. The invention is further refined by providing multiple points of guide wire fixation at the outer end of the positioning arm, so that multiple guide wires can be retained independently.

The invention is further refined to provide an adjustable positioning arm so that the operator can move the guide wire retention means to a variety of positions in space as may be perceived beneficial to the operator. Such adjustments can be accomplished using any of a variety of mechanisms including pivotable joints such as ball joints, sliding or telescoping rails, and bendable components.

The novel features of this invention, as well as the invention itself, will be best understood from the attached drawings, taken along with the following description, in which similar reference characters refer to similar parts, and in which:

### BRIEF DESCRIPTION OF THE SEVERAL VIEWS OF THE DRAWINGS

FIG. 1 is a perspective view of a first embodiment of the present invention, showing attachment to the luer fitting of the side port of a Y connector;

FIG. 2 is a perspective view of the embodiment shown in FIG. 1, with the positioning arm extended; and

FIG. 3 is a perspective view of a second embodiment of the present invention, showing attachment to the finger tabs on the body of a Y connector.

### DETAILED DESCRIPTION OF THE INVENTION

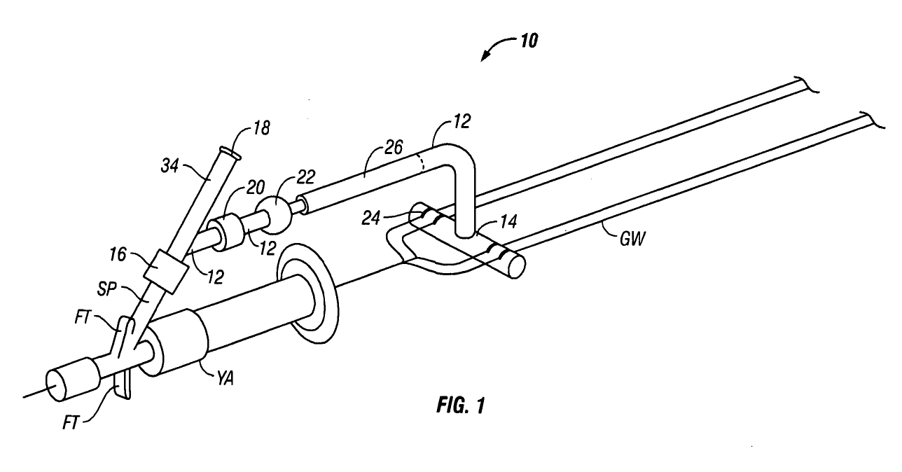

As shown in FIG. 1, the typical Y adaptor YA, as known in the art, can have one or more guide wires GW entering one end and exiting the other end. The Y adaptor YA seals around these guide wires GW, as described above, and as known in the art. The Y adaptor YA also has a side port SP which can have a luer fitting at its outer end. The luer fittings shown here are the luer lock type, but they could be the luer slip type. Finally, the Y adaptor YA may have one or more features such as finger tabs FT which are designed to facilitate the handling and operation of the Y adaptor. In the case shown, the finger tabs FT are formed on the body of the Y adaptor YA at the base of its side port SP.

The apparatus of the present invention 10 includes a guide wire positioning arm 12, which can have several sections, a guide wire retention mechanism 14, and a releasable attachment means 16 for attaching to the Y adaptor YA. In this case, the attachment means 16 consists of a female luer fitting for attaching to a male luer fitting at the outer end of the side port SP of a typical Y adaptor. The apparatus 10 can also have a male luer fitting 18 provided, such as at the outer end of a tubular conduit 34, to provide for connection of any desired fluid apparatus to the side port SP of a Y adaptor YA. When not in use, the male luer fitting 18 can be capped, as is known in the art. The guide wire positioning arm can be connected to the side of this tubular conduit 34. Alternatively, the guide wire positioning arm 12 could be connected directly to the side port SP by means of a luer fitting.

Sections of the guide wire positioning arm 12 can be connected together, such as by luer fittings 20, to promote versatility of the apparatus. It can be seen that the luer fitting 20 could be connected directly to the male luer fitting on the side port, if the tubular conduit 34 and its luer fitting 18 are not required for a particular procedure.

The positioning arm 12 can include a pivotable joint 22, such as a ball joint as shown or a universal joint. This allows the positioning arm 12 to extend straight from its connection to the tubular conduit 34 as shown, or to be oriented in any direction desired, by pivoting of the pivotable joint 22. The pivotable joint 22 can be constructed with sufficient stiffness to maintain the angle of the positioning arm 12 where positioned.

The positioning arm 12 can also include an extension means, such as a telescoping segment 26, allowing the positioning arm 12 to be extended in length as desired. The apparatus 10 is shown in FIG. 1 with the positioning arm in the retracted position. The guide wire retention mechanism 14 is shown as a T-shaped holder, which can include a longitudinal segment of an elastomeric material, or rubber, with a passive retention feature such as one or more slots 24 into which the guide wires GW can be inserted. Alternatively, the guide wire retention mechanism could comprise a spring actuated clamp, a slidable locking device, or a screw actuated clamp. In the embodiment shown in FIG. 1, the positioning arm 12 incorporates a right angle bend which positions the guide wire retention mechanism 14 in line with the main body of the Y adaptor YA. However, as mentioned above, the positioning arm 12 can be pivoted to position the guide wire retention mechanism 14 where desired.

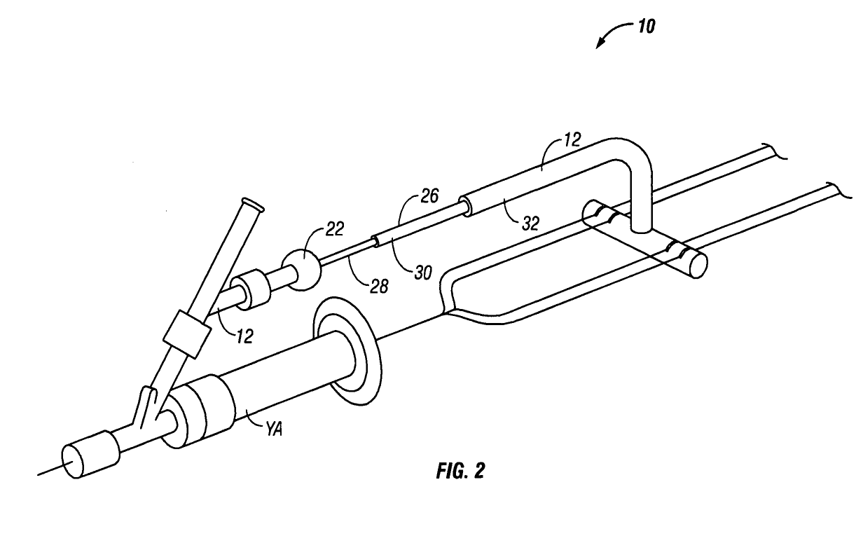

FIG. 2 shows the positioning arm 12 extended in length, which is accomplished by pulling the telescoping sections 28, 30, 32 outwardly to the desired length of extension. Of course, two telescoping sections, or more than three sections, could also be used.

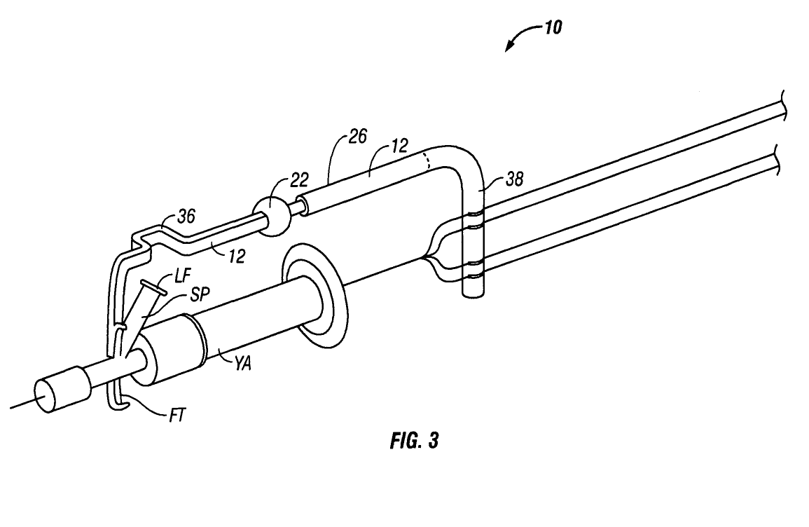

In the embodiment of the apparatus 10′ shown in FIG. 3, the positioning arm 12 is not attached to the outer end of the side port SP, but to the finger tabs FT at the base of the side port SP. This attachment is achieved by forming this end of the positioning arm 12 to fit tightly over the finger tabs FT, in a snap fit. This embodiment of the positioning arm 12 also incorporates a segment 36 which is bent to provide clearance for the side port SP of the Y adaptor YA. This segment 36 of the positioning arm 12 could be provided in a permanent shape to clear the side port SP, or it could be provided as a flexible segment of the positioning arm 12. Provision of a flexible segment 36 would enable routing of the positioning arm 12, either to clear the side port SP or to achieve any other positioning of the guide wire retention mechanism as may be required in any circumstance.

The embodiment shown in FIG. 3 also has a guide wire retention mechanism 38 which is straight, rather than T-shaped. Here again, the retention mechanism 38 can incorporate a rubber element having slots for retaining the guide wires. This embodiment is shown as including the pivotable joint 22 and the telescoping segment 26, but either or both of these features could be eliminated as required for a particular application. Further, the flexible segment 36 could be incorporated into the embodiment shown in FIGS. 1 and 2, in which the positioning arm 12 attaches to the luer fitting at the end of the side port SP of the Y adaptor YA. FIG. 3 shows the telescoping segment 26 in the retracted position, but it can be extended similar to the embodiment shown in FIG. 2. Similarly, the pivotable joint 22 can be pivoted to any desired orientation to position the retention mechanism 38 as desired.

While the particular invention as herein shown and disclosed in detail is fully capable of obtaining the objects and providing the advantages hereinbefore stated, it is to be understood that this disclosure is merely illustrative of the presently preferred embodiments of the invention and that no limitations are intended other than as described in the appended claims.

We claim:

1. An apparatus for retaining and positioning at least one guide wire extending from a sealable entry site of an intravascular Y adaptor, said retention and positioning apparatus comprising:
a guide wire positioning arm having a first end adapted for removable connection to said intravascular Y adaptor; and
a guide wire retention mechanism mounted to a second end of said guide wire positioning arm, said guide wire retention mechanism being adapted for releasable retention of said at least one guide wire.

2. The apparatus recited in claim 1, further comprising:
a tubular conduit, said guide wire positioning arm extending at an angle from said tubular conduit, said first end of said guide wire positioning arm being connected to a side of said tubular conduit; and
a tubular attachment mechanism on a first end of said tubular conduit, said attachment mechanism being adapted to connect said first end of said tubular conduit in fluid flow communication with a side port of said intravascular Y adaptor.

3. The apparatus recited in claim 2, wherein said tubular attachment mechanism comprises a luer fitting.

4. The apparatus recited in claim 2, further comprising a port on a second end of said tubular conduit.

5. The apparatus recited in claim 4, wherein said port on said second end of said tubular conduit comprises a luer fitting.

6. The apparatus recited in claim 1, further comprising a releasable attachment mechanism on said first end of said guide wire positioning arm, said releasable attachment mechanism being adapted for releasable connection to the body of said intravascular Y adaptor.

7. The apparatus recited in claim 6, said releasable attachment mechanism being further adapted for releasable connection at the base of a side port of said intravascular Y adaptor.

8. The apparatus recited in claim 1, wherein said guide wire retention mechanism comprises an elastomeric friction fitting.

9. The apparatus recited in claim 8, wherein said friction fitting comprises a slotted rubber member.

10. The apparatus recited in claim 1, further comprising an adjustment mechanism adapted for selective positioning of said guide wire retention mechanism in a desired position relative to said intravascular Y adaptor.

11. The apparatus recited in claim 10, wherein said adjustment mechanism comprises a pivotable joint.

12. The apparatus recited in claim 11, wherein said pivotable joint comprises a ball joint.

13. The apparatus recited in claim 10, wherein said adjustment mechanism comprises a telescoping section of said guide wire positioning arm.

14. The apparatus recited in claim 10, wherein said adjustment mechanism comprises a flexible section of said guide wire positioning arm.
